(19) United States
(12) Patent Application Publication
(10) Pub. No.: US 2010/0113957 A1
(43) Pub. Date: May 6, 2010

(54) SYSTEM, METHOD AND APPARATUS FOR MONITORING A MEDICAL CONDITION

(76) Inventors: Anthony Williams, Sydney (AU); Ronald Grunstein, Sydney (AU); Gunnar Unger, Sydney (AU)

Correspondence Address: RATNERPRESTIA, P.O. BOX 980, VALLEY FORGE, PA, 19482, US

(21) Appl. No.: 12/440,793

(22) PCT Filed: Sep. 11, 2007
(86) PCT No.: PCT/AU07/01342

§ 371 Date: Dec. 15, 2009

(30) Foreign Application Priority Data
Sep. 11, 2006 (AU) 2006905010

(51) Int. Cl. A61B 5/087; A61M 16/00

(52) U.S. Cl. 600/538; 128/204.18

(57) ABSTRACT

The present invention provides a device for use in monitoring a sleep apnoea condition, the device includes a cannula arranged to pass into a mask that is connectable to an airway pressure machine. The cannula may be utilised to administer the test while the mask is utilised to deliver airway pressure.

### FIELD OF THE INVENTION

The present invention relates to a system, method and apparatus for administering a medical test and/or for monitoring the treatment of a medical condition. The system is particularly, but not exclusively, useful in administering a sleep apnoea test.

### BACKGROUND OF THE INVENTION

The last twenty years has seen an exponential rise in the demand for medical services. This has placed an increasing strain (in terms of time and resources) on medical staff, hospitals, clinics and on governments, many of whom at least partially fund the health system.

Simultaneously, the advent of computer technology has resulted in medical devices that are more robust and sophisticated, and not only capable of collecting data, but also capable of performing at least part, if not all, of the analysis of the data. Such analysis would traditionally be performed by trained medical staff.

This has resulted in the advent of medical devices which may be used by patients to self-monitor or self-test. For example, blood pressure cuffs which self-inflate and provide a digital, easy to understand readout are now commonplace, and allow a patient to monitor their blood pressure without visiting a medical professional. Similarly, blood glucose testers have been developed, which allow a patient to self-test their blood glucose levels at any time.

As many patients do not fully understand how many medical devices operate and do not appreciate the finer points of how some medical devices should be used, it may be difficult for a patient to ascertain whether an unusual reading is the result of operator error, or whether the reading is a genuine cause for concern. For this reason, such devices must be arranged to minimise or eliminate the possibility of operator error.

In particular, in the area of sleep apnoea, many self-test devices and treatments are available. For example, to treat sleep apnoea, a Continuous Positive Airway Pressure (CPAP) machine is commonly used to delivering a constant stream of compressed air via a face mask and hose, splinting the airway (keeping the airway open by providing suitable air pressure) so that unobstructed breathing becomes possible, reducing and/or preventing apnoeas and hypopneas.

However, as the patient is asleep while using the machine, it is difficult for the patient to know whether the device is working adequately.

It will be understood that in the following specification, the term “CPAP machine” will be used to refer to any type of machine which operates to keep a patient's airway open during sleep to provide unobstructed breathing. This may include so-called “BiPAP” machines and “AutoPAP” machines, which vary in the manner in which air is delivered to the patient, but fundamentally utilise the same principle (i.e. keeping the patient's airway open by the use of compressed air).

### SUMMARY OF THE INVENTION

In a first aspect, the present invention provides a device for use in monitoring a sleep apnoea condition, comprising a cannula arranged to pass into a mask connected to an airway pressure machine capable of providing airway pressure to a patient, wherein the cannula may be utilised to administer the test while the mask is being utilised to deliver airway pressure.

In one embodiment, the device further comprises an adapter piece arranged to locate between the airway pressure machine and the mask, wherein the cannula is passed through an opening provided in the adapter piece.

The device may further comprise a nose piece arranged to connect to a first section of the cannula.

The nose piece may further comprise at least two openings arranged to locate adjacent to the nasal cavity of the patient, and at least two pads arranged to secure the nose piece to the nose of the patient.

The two openings may be prongs which extend at least partly into the nostrils of the patient.

There may be provided a wing attaching the pad to the nose piece, the wing being formed of a flexible material, wherein the pad may be moved relative to the prongs. The pads may further comprise a grip.

The nose piece may be integrally formed.

The cannula may further comprise a one way fitting arranged to connect the cannula to the device, wherein the one way fitting only allows one orientation of the cannula when it is fitted to the device.

The cannula may further comprise two sections. Where the cannula has two sections, each section of the cannula is in connection with at least two air pressure sensors in a monitoring device, the first sensor being arranged to measure the nasal airflow of a patient, and the second sensor being utilised to measure the air pressure outside of the patient's nasal cavity.

The monitoring device may be incorporated into an airway pressure machine.

In a second aspect, the present invention provides a nose piece arranged to engage with a cannula, comprising at least two openings arranged to locate adjacent to the nasal cavity of the patient, and at least two pads arranged to secure the nose piece to the nose of the patient.

The two openings may be prongs which extend at least partly into the nostrils of the patient.

The nose piece may include a wing attaching the each of the at least two pads to the nose piece, the wing being formed of a flexible material, wherein the wing is moveable to allow the each of the at least two pads to be moved relative to the prongs.

The each of the at least two pads may further include a grip portion.

### DETAILED DESCRIPTION OF THE DRAWINGS

Notwithstanding any other forms which may fall within the scope of the present invention, a preferred embodiment will now be described, by way of example only, with reference to the accompanying drawings in which:

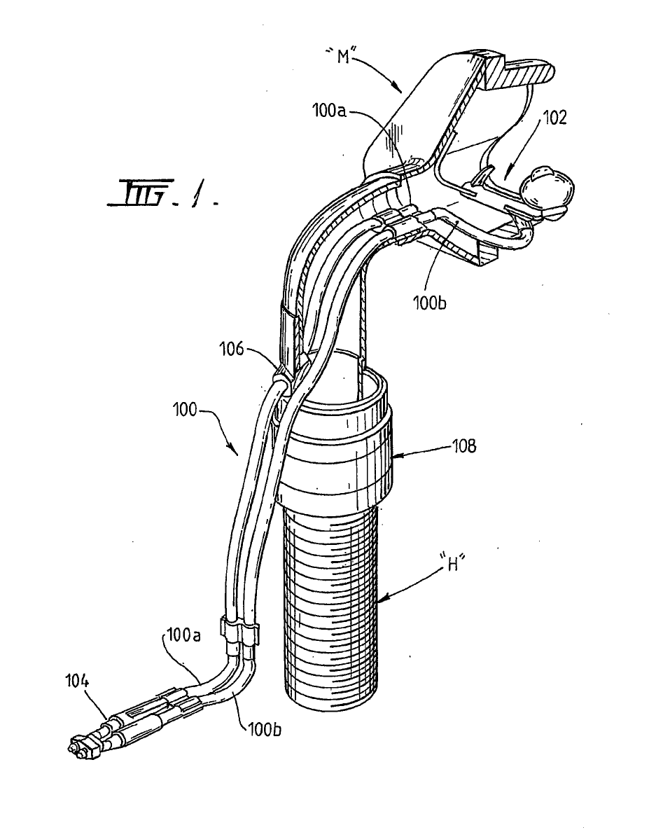

FIG. 1 is a diagram depicting a cannula, in situ, in accordance with an embodiment of the present invention;

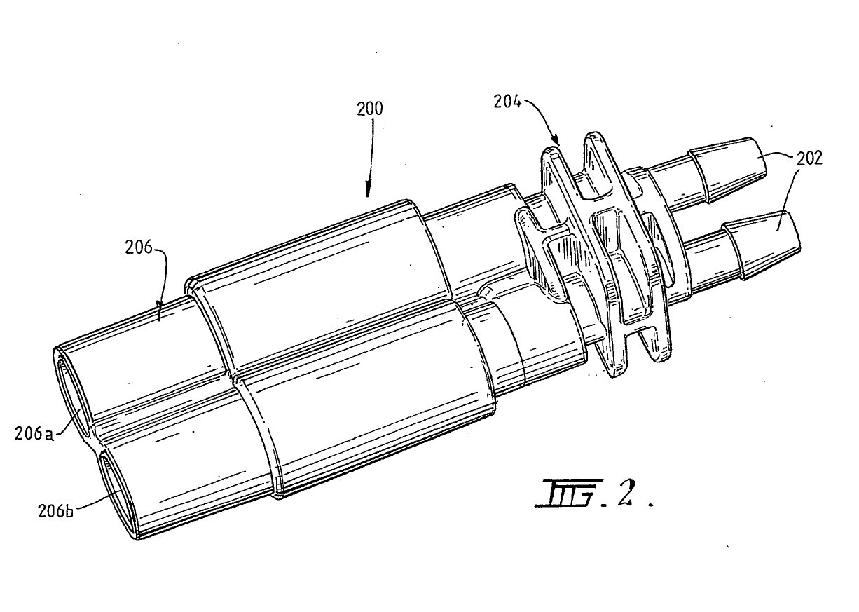

FIG. 2 is a diagram depicting a connection piece in accordance with an embodiment of the present invention; and

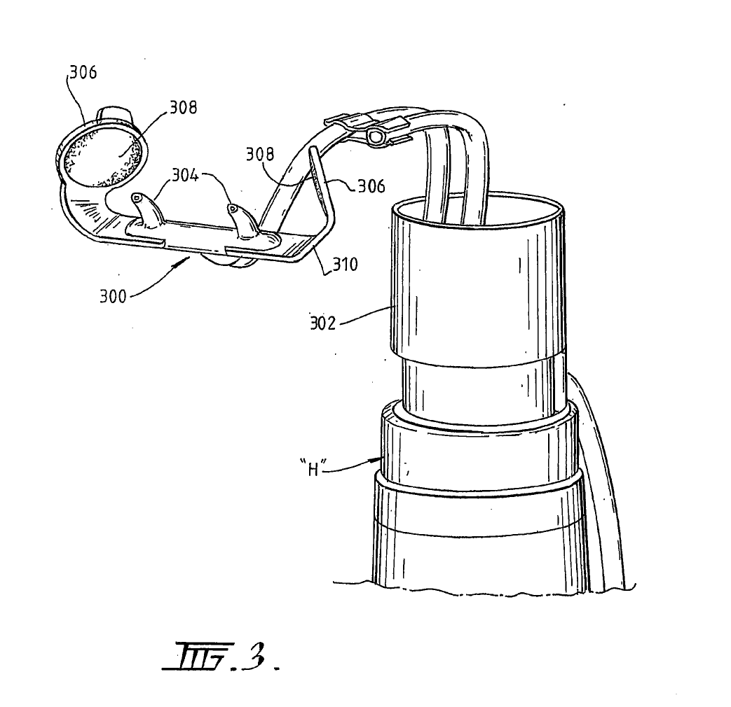

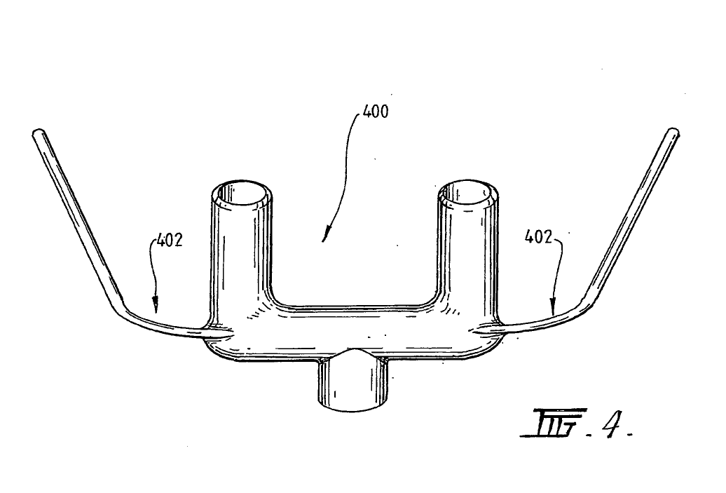

FIGS. 3 and 4 are diagrams depicting a nose piece in accordance with an embodiment of the present invention.

### DESCRIPTION OF SPECIFIC EMBODIMENTS

In the embodiment described herein, there is disclosed a medical device which can be used to administer a sleep apnoea test and/or monitor a previously diagnosed sleep apnoea condition. In particular, the embodiment is arranged to work in conjunction (i.e. simultaneously) with a CPAP (or equivalent) machine.

In one embodiment, the device includes a dual pipe cannula 100 which includes a nose piece 102 and a connection piece 104 (to connect the cannula to the device). The cannula 100 is designed to be utilised in conjunction with (i.e. integrate with) a CPAP machine mask.

The dual pipe cannula 100 allows for two different air pressures (flow is derived from the pressure in the nasal cavity) to be measured. The first pipe 100a is arranged to open directly into the mask region, while the second pipe 100b is arranged to be connected to the nose piece 102. It will be understood that while the present embodiment incorporates two separate tubes, the cannula 100 may be comprised of two integrally formed tubes, or it may be comprised of a single tube which includes a dividing wall to form two channels. Such variations are within the contemplation of a person skilled in the art.

In more detail, the cannula 100 is arranged to pass through an opening 106 in an adapter piece 108 which may be placed between the CPAP machine hose “H” and the CPAP machine mask “M” in order to access the patient's nose.

The adapter piece 108 allows the cannula 100 to be integrated into any existing CPAP machine and it will be understood that the adapter piece may be made in different sizes and/or shapes to suit different types of CPAP machines.

The cannula further includes a connection piece 104 arranged to connect the cannula to the device. The connection piece 104 is shown in more detail in FIG. 2.

Referring to FIG. 2, the connection piece 200 (equivalent to connection piece 104 in FIG. 1) includes dual prongs 202 arranged to connect to the dual pipe cannula. The connection piece 200 also includes a grip 204 arranged to provide assistance in pressure fitting the cannula to the connection piece. That is, a user may use the grip 204 to hold the connection piece steady while pressing the cannula onto the connection piece the grip 204 also has the attendant advantage of being designed to fit into and be secured by the recorder's plastic packaging. The space into which the ‘Y site’ connector will fit is currently occupied by a single luer lock. As the device requires two channels (for the two pipes), yet the device only has one inlet, the grip 204 allows the connector to be secured into the device while allowing for cost effective tooling.

The connection piece 200 further includes a port fitting 206 arranged to connect the connection piece 200 to the device. The port fitting includes two differently sized ports 206a and 206b. The differently sized port fittings prevent a user from accidentally connecting the cannula incorrectly to the device.

In other words, by utilising different sized ports, the user does not need to consider which pipe (or port fitting) should be connected to a given port on the device. This prevents confusion, makes setup easier, and removes the possibility of erroneous readings due to an incorrect setup.

Referring to FIG. 3, there is shown a nose piece 300 (which is equivalent to the nose piece 102 shown in FIG. 1). The nose piece 300 is shown in situ, as it would appear when passed through a CPAP machine hose “H” through an adapter piece 302. The nose piece includes two nasal prongs 304 arranged to fit within the nasal cavity of a patient. There is also included a pair of holding pads 306 arranged to hold the nose piece 300 in place on a patient's nose, despite the piece being subjected to normal forces caused by patient movement. Each holding pad 306 may include appropriate non-slip grips 308 to prevent the pad 306 from slipping. The grips 308 may be composed of any suitable material such as rubber or a plastics material, which simultaneously prevent slippage, while being comfortable to the touch. The grip may also include a rough surface (which may be provided by adding nodules, depressions, or other suitable patterns to the grip) to assist in holding the grip to the nose of a patient.

Each pad 306 may be fastened to the nose piece 300 via a wing 310 of flexible material, arranged to allow the pad to be moved through various angles relative to the prongs 304. This allows the nose piece 300 to be adjusted to fit differently sized and shaped noses.

Referring to FIG. 4, there is shown a nose piece 400 (equivalent to nose piece 300 of FIG. 3) which illustrates a side view of wings 402. The placement of the wings 402 in this manner provides maximum flexibility, while also being designed to be comfortable for the patient.

The nose piece 300 of FIGS. 3 and 400 of FIG. 4 are integrally formed in one piece, from a medical grade material, and are ideally resistant to bacterial growth. However, it will be understood that the nose piece may be formed from any suitable material. It will also be understood that the nose piece may also be formed as a number of distinct components, and subsequently joined together either by the pressure fitting of parts, plastic welding, gluing, or any other suitable method. Such variations are within the contemplation of a person skilled in the art.

The device is in communication with a system capable of measuring and accurately recording breathing patterns. The system, which is described here to further illustrate the device as a whole, includes a microcontroller which may be an Atmel™ Model No. ATMEGA32L-8MC. The microcontroller is capable of receiving two analogue signals sampled at 2000 Hz, calculate the mean of the two signals, and store the data collected memory at a rate of 25 Hz per channel.

In addition, the Microcontroller is capable of deriving, from one of the analogue signals, the difference between the maximum and minimum values for 240 samples, and store these values separately.

The device also includes an analogue to digital converter which may be an integrated part of the microcontroller. The Analogue to digital converter is capable of providing 10 bit resolution.

The microcontroller (and analogue to digital converter) receive raw data from two separate pressure transducers. The first is arranged to measure nasal flow, (which may be derived from a measurement of nasal pressure). The second is arranged to measure mask pressure. In a typical application, the nasal pressure range is ±150 Pascal, so a pressure transducer with a range of ±1000 Pascal provides a more than adequate range. The transducer is connected to two connecting tubes, the first providing air for measurement of nasal pressure and the second providing measurement of mask pressure.

The typical mask pressure range is in the order of 1600 Pascal (from 400 Pascal to 2000 Pascal) (which is always positive, as it is assumed that the mask is fed a continuous or cyclical positive pressure by virtue of being connected simultaneously to a CPAP machine). Only one tube is connected to the second transducer, as the reference point is atmospheric pressure.

In another embodiment, nasal flow may be measured for each nostril separately, by providing a low pressure range transducer in place of the mask pressure sensor. This transducer has the same pressure range. Sensitivity is also the same for both transducers.

Signals from the pressure transducers are amplified via a DC amplifier. In addition the mask pressure channel has software calibration capabilities, to create a linear calibration curve between 0 and about 2000 Pascal.

The aforementioned memory is capable of storing data collected during a defined time period, such as 27 hours, with 2 channels at 25 Hz (min 10 bits) and 1 channel at 8.33 Hz (8 bits).

It will also be understood that the device may include any other components necessary for appropriate operation, including a power source (such as a battery), a communications interface (for example, a USB connection, an Ethernet or an

Infra-red connection), appropriate controls (for example, a power switch) and LED or LCD displays capable of displaying the status of the device.

The device is operated by an EPROM or similar hard (or soft) wired control system. The operating system includes the capability of providing a redundancy check on the data (including the identifying parameters) so that integrity can be assured from measurement through to the report. This may be a CRC (cyclic redundancy check). Moreover, the device may be capable of holding a number of changeable or settable parameters, including but not limited to:
A settable Zero level for nasal flow and mask pressure;
A setting to Calibrate mask pressure;
A Device ID;
A Patient ID;
A real time clock to record the start time of recording, and the duration of recording; and
A counter to count the number of recordings.

It will be understood that such settings, and the firmware as a whole may be updated or replaced via the communications link.

The device may also be capable of recording user event marks (for example, the time at which a button is pressed by a user), which may have a later bearing on the data or the analysis performed by the device.

The two pressure transducers are capable of reading nasal flow, derived from nostril pressure, and mask pressure and recording the data at 25 Hz with 10 bits resolution.

Snore signals may also be recorded and stored. Nasal Flow and Snore recordings may be utilised in conjunction with the Epworth sleepiness score and the patients BMI (body mass index) to more accurately determine a reason for the patient's sleepiness. In other words, the device is capable of recording and storing the following types of medical data:
The number of apnoeas (including start and duration time);
The number of hypopneas (including start and duration time);
The number of flow limitations (including start and duration time);
The duration of NOT acceptable quality signals;
The duration of two levels of snoring; and
The corresponding mask pressure during the events.

This information may then be downloaded and produced in a report format for further analysis and diagnosis, or alternatively, preliminary analysis may be carried out by the device.

The preliminary analysis can be performed by determining the flow amplitude and limitation then applying a rule set to determine the category of the flow.

For example, if the flow amplitude was reduced during a recorded event to less than 10% of the typical or normal flow, and the reduction lasted from a minimum of 10 seconds to a maximum of 2 minutes, then an apnoea has occurred.

In another example, if the flow amplitude was reduced during a recorded event to less than 50% of the typical or normal flow, and the reduction lasted from a minimum of 10 seconds to a maximum of 2 minutes, then a hypopnoea has occurred.

A breath with snore may be detected by taking the difference between maximum and minimum pressure levels, at a high sample rate, such as 2000 Hz. When the amplitude of the high frequency signal is greater than a set level, then the breath is marked as a snore event. A higher level will indicate a more intense snoring event.

Where the device is performing preliminary analysis, it is also desirable to include a number of functions which automatically analyse data and either exclude data or vary the data if detectable problems are found.

For example, the device will exclude (or erase) data where no there is no detectable flow (for example, at the beginning or at the end of a recording, or where a reading has been taken where the cannula is not in place).

Moreover, there may be DC drift in the flow signal due to temperature changes. As the drift may occur over a long time period, adjustment may only be required every given time period, such as every 10 minutes. Such adjustment may be done between breaths (i.e. when a person momentarily pauses between breaths)

The analysis may also need to ascribe a sensible cause to a sudden large variation in the flow signals amplitude. In the embodiment described, analysis will continue while the flow signals amplitude remains detectable. In the case of dislocation of a cannula, mouth breathing, etc. there will be a lack of a flow signal longer than 2 minutes, which in turn will trigger a “not acceptable signal” event, which will be registered in the memory.

Moreover, the device may be arranged to provide a warning if it believes that the collected data set is poor (unreliable) data. Poor data is defined as data where the moving average of 20 breaths is below a set value. The value is set to a level where it is still possible to detect variation in the flow signal. This can be indicative of an apnoea or hypopnoea.

The embodiment described herein provides a number of advantages. Firstly, the embodiment is arranged to operate in conjunction with a CPAP machine. This allows a patient (and/or a medical professional) to monitor, in a real time and ongoing basis, the performance of the CPAP machine. In turn, this allows the patient and/or medical professional to adjust the CPAP machine to provide the best possible outcome for the patient.

Secondly, the embodiment utilises a cannula which may only be fitted in one way, thereby preventing the patient from making an error in the setup of the device. This reduces the risk of operator error when the device is used by a patient with little experience.

Thirdly, the embodiment is designed to fit into an existing CPAP machine without causing any leak from or into the CPAP mask. The mask associated with the CPAP machine continues to fit under the nose of a patient. The embodiment provides a system which, while allowing for an interface between a patient and a sleep related breathing test device, doesn't impact on the treatment provided by the CPAP machine and has minimal effect on the sleep quality of the patient.

Fourthly, the embodiment allows the recording of two pressure signals while maintaining comfort and compliance over and above standard nasal oxygen cannulas that are used for oxygen delivery.

It will be understood that whilst the embodiment described herein is utilised to monitor a sleep apnoea condition, the embodiments described herein may find use in other medical tests.

1. A device for use in monitoring a sleep apnoea condition, comprising a nose piece arranged to connect to a cannula, the cannula being arranged to pass into a mask connected to an airway pressure machine capable of providing airway pressure to a patient, wherein the cannula may be utilised to administer the test while the mask is being utilised to deliver airway pressure.

2. A device in accordance with claim 1, further comprising an adapter piece arranged to locate between the airway pressure machine and the mask, wherein the cannula is passed through an opening provided in the adapter piece.

3. A device in accordance with claim 1, wherein the nose piece is arranged to connect to a first section of the cannula.

4. A device in accordance with claim 3, wherein the nose piece further comprises at least two openings arranged to locate adjacent to the nasal cavity of the patient, and at least two pads arranged to secure the nose piece to the nose of the patient.

5. A device in accordance with claim 4, wherein the two openings are prongs which extend at least partly into the nostrils of the patient.

6. A device in accordance with claim 4, further comprising a wing attaching the pad to the nose piece, the wing being formed of a flexible material, wherein the pad may be moved relative to the prongs.

7. A device in accordance with claim 4, wherein the pads further comprise a grip.

8. A device in accordance with claim 4, wherein the nose piece is integrally formed.

9. A device in accordance with claim 2, wherein the cannula further comprises a one way fitting arranged to connect the cannula to the device, wherein the one way fitting only allows one orientation of the cannula when it is fitted to the device.

10. A device in accordance with claim 1, the cannula further comprising two sections.

11. A device in accordance with claim 10, wherein the two sections of the cannula are in connection with at least two air sensors in a monitoring device, the first sensor being arranged to measure the nasal airflow of a patient, and the second sensor being utilised to measure the air pressure outside of the patient's nasal cavity.

12. A device in accordance with claim 11, when incorporated into an airway pressure machine.

13. A nose piece arranged to engage with a cannula, comprising at least two openings arranged to locate adjacent to the nasal cavity of the patient, and at least two pads arranged to secure the nose piece to the nose of the patient.

14. A nose piece in accordance with claim 13, wherein the two openings are prongs which extend at least partly into the nostrils of the patient.

15. A nose piece in accordance with claim 13, further comprising a wing attaching the each of the at least two pads to the nose piece, the wing being formed of a flexible material, wherein the wing is moveable to allow the each of the at least two pads to be moved relative to the prongs.

16. A nose piece in accordance with claim 13, wherein the each of the at least two pads further include a grip portion.

17. A nose piece in accordance with claim 13, wherein the nose piece is integrally formed.
